(19) United States
(12) Patent Application Publication
(10) Pub. No.: US 2019/0198143 A1
(43) Pub. Date: Jun. 27, 2019

(54) DRUG COMPLIANCE SYSTEM AND METHOD

(71) Applicant: Hydraceil LLC, Garnet Valley, PA (US)

(72) Inventors: William L. Corcoran, Garnet Valley, PA (US); Robert E. Wallace, Newtown Square, PA (US); Christine Meyer, Exton, PA (US)

(21) Appl. No.: 15/854,366

(22) Filed: Dec. 26, 2017

(51) Int. Cl. G16H 20/10; A61B 5/00; A61J 7/00

(52) U.S. Cl. CPC G16H 20/10; A61B 5/4833; A61J 1/03; A61J 2200/30; A61J 7/0076

(57) ABSTRACT

A method of determining a patient's compliance with a prescription, the prescription indicating the dosage of prescription pills to be consumed in a certain period, the method comprising: (a) receiving an image of one or more remaining pills from a patient on a given date, the given date being after an initial date when the patient received an initial number of the prescription pills; (b) confirming that the remaining pills are the same type as the prescription pills; (c) determining whether the number of the remaining pills corresponds with an expected number of remaining pills, the expected number of remaining pills being the initial number minus a calculated number of pills taken between the initial date and the given date based on the dosage; and (d) generating a report of the patient's compliance based on whether the number of the remaining pills corresponds to the expected number of remaining pills as determined in step (c).

This application is a utility conversion of U.S. Provisional Application No. 62/438,616, filed Dec. 23, 2016, which is hereby incorporated by reference in its entirety.

### FIELD OF INVENTION

This disclosure relates generally to compliance systems for controlled substances, and, more specifically, to computer-implemented compliance systems and method for monitoring a patients consumption of drugs to minimize the opportunity for abuse.

### BACKGROUND

Drugs are an indispensable component of healthcare. Drugs are used to treat disease, fight infection, and manage pain and anxiety, among other important uses. A statistically significant amount of drugs used to treat pain and anxiety are subject to abuse. The United States has 5% of the world's population yet consumes 75% of the world's prescription drugs. Over two million people in the United States suffer from substance use disorders related to prescription opioid pain relievers. The terrible consequences of this epidemic substance abuse include overdose deaths, which have more than quadrupled in the past decade and a half Over a hundred people die from drug overdoses every day in the United States. More people die from overdoses of prescription opioids than from all other drugs combined, including heroin and cocaine.

Due to their tendency for abuse, the U.S. government, along with other foreign governments, has deemed such drugs controlled substances. Drugs, substances, and certain chemicals used to make drugs are classified into five (5) distinct categories or schedules depending upon the drug's acceptable medical use and the drug's abuse or dependency potential. The abuse rate is a determinate factor in the scheduling of the drug. For example, Schedule I drugs are considered the most dangerous class of drugs with a high potential for abuse and potentially severe psychological and/or physical dependence. Schedule I drugs have no medically approved purpose. Next, Schedule II through V drugs generally have an approved medical purpose. As the schedule increases, from Schedule II to Schedule V, the potential for abuse decreases, with Schedule V drugs representing the least potential for abuse. A listing of drugs and their schedule are located at Controlled Substance Act (CSA) Scheduling or CSA Scheduling (incorporated by reference)

Of particular interest herein are Schedule II and III drugs, which are the most commonly abused pharmaceutical drugs. It should be noted, however, that the scheduling of a drug is not a requirement for its consideration herein. Schedule II drugs, substances, or chemicals are defined as drugs with a high potential for abuse with use potentially leading to severe psychological or physical dependence. In light of the potential for abuse, these drugs are considered dangerous. Examples of Schedule II drugs include cocaine, methamphetamine, methadone, hydromorphone (Dilaudid), meperidine (Demerol), oxycodone (OxyContin), fentanyl, Dexedrine, Adderall, and Ritalin. Schedule III drugs, substances, or chemicals are defined as drugs with a moderate to low potential for physical and psychological dependence. Examples of Schedule III drugs include combination products with less than 15 milligrams of hydrocodone per dosage unit (Vicodin), products containing less than 90 milligrams of codeine per dosage unit (Tylenol with codeine), ketamine, anabolic steroids, and testosterone.

Schedule II and III drugs are known to be abused by the patients to whom the drugs are prescribed. It is well known that a significant number of patients consume far more pills than the prescribed dosage. It is not unusual for patients using prescription drugs to take 5 to 10 times, or more, of the prescribed dose. The resulting effects of both psychological and physiological dependency are well documented in patients suffering from drug abuse. There are also several detrimental side effects of such drugs, including liver damage, digestion problems, and other ailments. Another less obvious result of abusing prescription drugs is that the patient who abuses them tends to consume the entire prescription soon after it is filled, and must suffer with pain or anxiety until the prescription can be filled again, which may be weeks later.

Various approaches to curb drug abuse have been tried. For example, one approach involves scheduling the drugs as described above. Specifically, prescriptions for Schedule II drugs cannot be refilled, and thus require the patient to see the physician each time the prescription needs to be refilled, allowing the physician to assess the patient and determine compliance. Additionally, the logistics of a patient seeing the doctor for each refill tends to control the amount of drugs the patient has available. Likewise, limitations are placed on Schedule III drugs in connection with the number of refills allowed, thereby reducing abuse for the reasons mentioned above. This approach, however, places an administrative burden on physicians and other health care professionals, and is, by design, an inconvenience to the patients.

Furthermore, in an attempt to curb abuse of Schedule II drugs, “combination drugs” (which are schedule III drugs) were developed which combine Schedule II drugs, such as oxycodone or hydrocodone, with secondary drugs, such as acetaminophen (Tylenol), which is non-additive, to restrict the amount of Schedule II drugs a patient can take. For example, Percocet is a combination of oxycodone (Schedule II) and Acetaminophen. Drugs like Acetaminophen (Tylenol) have maximum daily dosages that the body can handle (many Schedule II drugs have no such limit). For example, Tylenol has a ceiling of 4 grams per 24-hour period. Accordingly, prescriptions are written for combination drugs such that the amount of Schedule II drug cannot exceed the ceiling of a secondary drug with which it is combined. One objective of combination drugs is that the secondary drug will serve to curb consumption. Although this method ensures that the prescription is limited by virtue of the ceiling of the secondary drug, often the patient abuses the drug anyway, and the side effects of the secondary drug present significant health risks to the patient in the event of overdose. For example, Tylenol is detrimental to the liver if its ceiling of 4 grams per day is exceeded. Therefore, even though combination drugs have limited dosage to comply with the secondary drug's ceiling, often the drug is nevertheless abused, resulting in not only drug dependency, but also detrimental side effects such as liver damage or even death from the secondary drug.

In a recent development, many Schedule III combination drugs are being reclassified as Schedule II. Moreover, due to the dangers of the combination medication (i.e., Acetaminophen), many combinations drugs (e.g. Percocet, Vicodin, and Lortab) are slated to be discontinued or banned due to their role in deadly overdoses).

Often, due to the liability resulting from abuse, physicians simply refuse to prescribe such medication. This can result in patients receiving more costly medication with increased side effects. Additionally, refusing to prescribe these drugs can prevent patients from receiving the therapy they need.

Therefore, Applicants identify the need for a compliance system which monitors possible abuse, but does not impose onerous regulatory/administrative tasks on the prescriber. The present invention fulfills this need among others.

### SUMMARY OF INVENTION

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

The present invention relates to a compliance monitoring system that recognizes that all pills have a unique physical appearance, and that, with today's technology, patients have the ability to take a picture of the pills they have on hand and to transmit the image almost instantly. Thus, Applicants have devised a prescription medicine compliance monitoring system that determines whether a patient is compliant by receiving an image of the patient's remaining prescription pills, by verifying that the remaining pills imaged are indeed the pills prescribed based on their unique appearance, and then comparing the number of remaining pills in the image to the expected number of remaining pills based on the patient's dosage to determine if the proper amount of pills have been taken according to the prescription.

One aspect of the invention is a method for monitoring compliance. In one embodiment, the method comprises: (a) receiving an image of one or more remaining pills from a patient on a given date, the given date being after an initial date when the patient received an initial number of a prescription pills; (b) confirming that the remaining pills are the same type as the prescription pills; (c) determining whether the number of the remaining pills corresponds with an expected number of remaining pills, the expected number of remaining pills being the initial number minus a calculated number of pills taken between the initial date and the given date based on the dosage; and (d) generating a report of the patient's compliance based on whether the number of the remaining pills corresponds to the expected number of remaining pills as determined in step (c).

Another aspect of the invention is a system for monitoring compliance. In one embodiment, the system comprises: at least one processor; one or more data stores operatively connected to the processor; a network interface to transmitting requests and reports; and memory operatively connected to the process and configured with instructions for causing the processor to execute the following steps: (a) receiving an image of one or more remaining pills from a patient on a given date, the given date being after an initial date when the patient received an initial number of the prescription pills; (b) confirming that the remaining pills are the same type as the prescription pills; (c) determining whether the number of the remaining pills corresponds with an expected number of remaining pills, the expected number of remaining pills being the initial number minus a calculated number of consumed pills based on the dosage taken between the initial date and the given date; and (d) generating a report of the patient's compliance based on whether the number of the remaining pills corresponds to the expected number of remaining pills as determined in step (c).

Yet another aspect of the invention is software which can be downloaded and used to configure a patient's device to cooperate with the system above. In one embodiment, the patient's device comprises: a camera for taking an image of pills; an network interface for transmitting the image over a network to a compliance system; and a user interface configured to instruct the user when to take an image of the pills.

### BRIEF DESCRIPTION OF DRAWINGS

FIG. 1 is a flow chart of one embodiment of the method of the present invention.

FIG. 2 is a schematic of one embodiment of the system of the present invention.

### DETAILED DESCRIPTION

The term “controlled substance” as used in this application is defined broadly as any drug or pharmaceutical that is subject to abuse. Typically, although not necessarily, controlled substances will be Schedule II or III substances set forth in the Controlled Substance Act (CSA). Of particular interest herein are controlled substances in the form of solid, discrete units, such as, pills, capsules, and tablets. For convenience, these units are referred to herein as “pills,” collectively.

The term “prescription” is broadly defined as a communication of any type (e.g., email, electronic (e-prescription), paper, oral communication, telephone call, facsimile, xml etc.) in which a prescriber designates a dosage of a controlled substance for a particular patient. The dosage is the amount of pills to be taken by a patient within a certain time. For example, the dosage may be 10 mg of oxycodone every 4 hours. The prescription may also set forth a number of dosages and/or a duration over which the dosages should be taken. Although a prescription may be any communication setting forth the dosage as described above, a prescription is typically issued by a prescriber having authority to issue prescriptions for controlled substances. Prescribers include, for example, physicians, nurse practitioners, psychologists, podiatrists, and other healthcare professionals. The person to whom the prescription is prescribed is referred to herein as the “patient” regardless of whether the person is under the medical supervision of a health care professional.

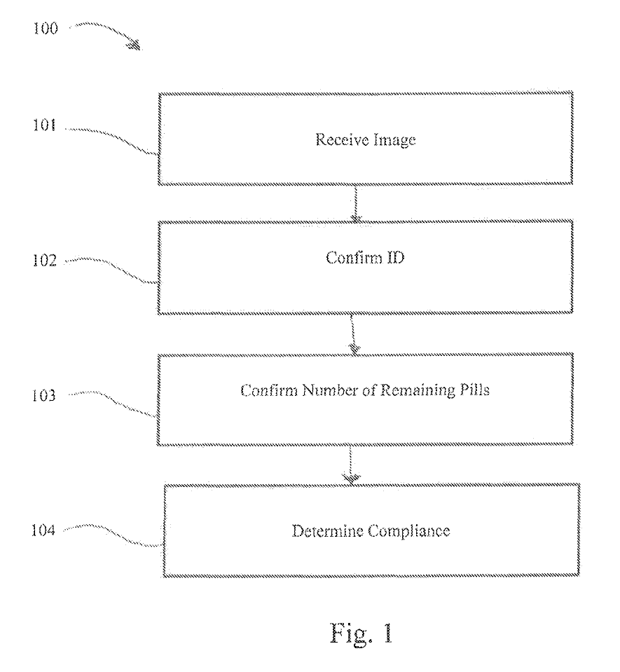

Referring to FIG. 1, a flow chart is shown of a method embodiment of the invention of determining a patient's compliance with a prescription, in which the prescription indicating the dosage of prescription pills to be consumed in a certain period. In Step 101, the system receives an image of one or more remaining pills from a patient on a given date, the given date being after an initial date when the patient received an initial number of the prescription pills. In step 102, the system confirms that the remaining pills are the same type as the prescription pills. In step 103, the system determines whether the number of the remaining pills corresponds with an expected number of remaining pills, the expected number of remaining pills being the initial number minus a calculated number of consumed pills based on the dosage between the initial date and the given date. In step 104, the system generates a report of the patient's compliance based on whether the number of the remaining pills corresponds to the expected number of remaining pills as determined in step 103.

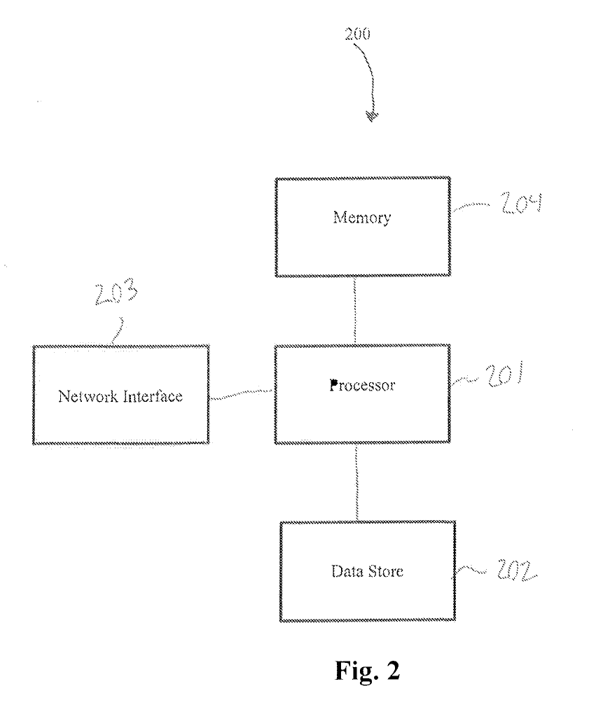

Referring to FIG. 2, a schematic of one embodiment of a system 200 for monitoring compliance is shown. As shown, the system comprises at least one processor 201, one or more data stores 202 operatively connected to the processor, a network interface 203 to transmitting requests and reports, and memory 204 operatively connected to the process and configured with instructions for causing the processor to execute the following steps: (a) receiving an image of one or more remaining pills from a patient on a given date, the given date being after an initial date when the patient received an initial number of the prescription pills; (b) confirming that the remaining pills are the same type as the prescription pills; (c) determining whether the number of the remaining pills corresponds with an expected number of remaining pills, the expected number of remaining pills being the initial number minus a calculated number of consumed pills based on the dosage taken between the initial date and the given date; and (d) generating a report of the patient's compliance based on whether the number of the remaining pills corresponds to the expected number of remaining pills as determined in step (c).

It should be understood, however, that the schematic of FIG. 2 is provided for illustrative purposes only, and the system and process of the present invention may be practiced in ways not specifically shown in FIG. 2. For example, although certain components and systems are depicted as single entities, this is done for illustrative purposes only. It should be understood that these components may be integrated into a single device such as, a server, or the functionality of the components may be distributed over a network. Additionally, it should be understood that the various components shown in FIG. 2 are not necessarily housed in a common area or even operated by a common entity—i.e., the various components may be operated by different companies and interfaced together. For example, in one embodiment, the data store function and the processing functions are executed in the Cloud. In this embodiment, the patient/user may use an ordinary smart phone having a camera to capture an image of the pills and transmit the image to the cloud for processing and compliance determination. From the cloud, one or more interested parties can receive information on the patients compliance. Therefore, the schematic of system 200 should not be used to limit the structure of the system more narrowly than the claims.

The embodiments of FIG. 1 and FIG. 2 are discussed in detail below.

In step 101, the compliance system receives an image of one or more remaining pills from the patient on a given date after an initial date when the patient received an initial number of the prescription pills. As used herein, the “initial date” is the date (and possibly time) that the patient receives the prescription. This can be entered into the system in different ways. For example, in one embodiment, the system of the present invention is integrated with state or federal prescription monitoring programs (PMPs) or prescription drug monitoring programs (PDMPs). Such programs are well known. These programs collect and distribute data about the prescription and dispensation of federally controlled substances and, as the individual states deem appropriate, other potentially addictive or abusable prescription drugs. Pharmacies that dispense controlled substances and providers who prescribe them are typically required to register with their respective state PMPs and (for pharmacies and providers who dispense controlled substances from their offices) to report the dispensation of such prescriptions to an electronic online PDMP database. Thus, prescriptions are monitored by the PDMP and the pharmacy or drug dispensary enters the time a prescription is filled into the PDMP database. This time is the initial date as used in this disclosure.

In another embodiment, the system 200 does not integrate with the PDMP, but operates independently with a proprietary database. In this embodiment, when the prescription is filled, the pharmacy or drug dispensary enters the initial date the prescription is filled into the proprietary database. In yet another embodiment, the patient is trusted to enter the initial date into a database. Still other embodiments will be obvious to those of skill in light of this disclosure.

The patient may or may not be prompted by the compliance system to transmit the image. In one embodiment, the system prompts the patient to transmit the image of the remaining pills either periodically or randomly. Generally, although not necessarily, it may be preferable to prompt the patient randomly to avoid any manipulation of the system. The frequency of the transmission requests may depend on a number of factors, including, for example, the patient's history of drug abuse and the potency of the drug being prescribed. For example, if a patient is known to have had an addiction problem or otherwise abuse prescription medicines or if the medication being prescribed is particularly potent, such as, OxyContin, then the system may prompt the patient to transmit images of the remaining pills on a frequent basis, for example, once every couple of days or even more frequently. On the other hand, if the drug being prescribed is relatively benign, for example, Ritalin, or the patient has no previous history of abusing medication, then the frequency of the image transmissions may be much less, for example, once every few months of so. In one embodiment, the frequency of the prompts is determined by an algorithm which is based upon the patient's data including their medical history and the potency of the drug which is stored in the prescription data.

Alternatively, rather than the system prompting the patient to transmit the image of the pills, the patient may, without prompting, transmit the image of the pills to the system for a determination of compliance. In other words, the patient may be interested in determining whether he or she may have accidently missed or exceeded a dosage. In this respect, it is generally well known that the pill-taking regimens of the chronically ill or elderly can be very complex, often involving multiple prescriptions and the consumption of many different pills throughout the day. Although systems, such pill boxes with the days of the week indicated on them, have been used to help patient compliance, it nevertheless is a daunting task for many, and mistakes are often inevitable. Rather than risking over-medication or under-medication, if the patient is unsure of their compliance, the patient can transmit an image of the remaining pills to the system to determine whether or not they need to take the pills at that time. Still other methods for other reasons for using the system to determine compliance will be known to those of skill in the art in light of this disclosure.

The patient may transmit the image in a number of different ways. Generally, it is preferred, although not necessary, that the image be transmitted without delay, and thus reflects a contemporaneous accounting of the number of remaining pills the patient has at the time the image was taken. Accordingly, in one embodiment, the image is taken and transmitted using a wireless device. Suitable wireless devices include, for example, smart phones and tablets. Alternatively, the device can be a dedicated apparatus for taking and transmitting the image, and may include, for example, a kiosk in a public area, such as in a drug store. Still other means of taking and transmitting an image of the patient's remaining pills will be known to those of skill in art in light of this disclosure.

As mentioned above, it is generally preferred that the transmitted image represents a contemporaneous accounting of the pills in the patient's position. Thus, in one embodiment, the image is date stamped such that the time between when the prescription was filled and the date of the image can be determined with certainty. The use of date-stamps is well-known. Generally, a date-stamp application is desired that is difficult to manipulate to ensure integrity in the system. Accordingly, in one embodiment, imaging involves the use of a special application which date-stamps the image that is transmitted to the system. In one embodiment, the application is communicatively linked with the compliance system to alert the patient when to transmit an image and which pills to image. This imaging application may be readily down-loaded from the Internet onto a smart phone or tablet.

Alternatively, rather than date-stamping the image, a request can be sent to the patient to manipulate the image of the pills in a certain way as to ensure that the image was taken after the particular request was made. For example, in one embodiment, the compliance system transmits a request to the patient to arrange the pills in the image a certain shape (e.g., circle, square, triangle, etc.), thus ensuring that the image was taken after the request was transmitted. Likewise, the request can contain instructions to include in the image other randomly chosen, but commonly-found objects in a house, e.g., a pencil, paper clip, coffee mug, etc. In another embodiment, the perspective of the remaining pills can be changed, e.g., top view, side view, etc. In yet another embodiment, the date of the image may be confirmed by having the patient image the pills with a date sensitive object in the picture, such as, for example, a newspaper or a television screen. Still other techniques for attributing a date to a particular image would be known to those of skill in in the art in light of this disclosure.

In Step 102, the system confirms whether the remaining pills in the image are the prescription pills of the prescription. In this respect, it should be understood that prescription pills have a unique shape, size and color configuration such that the pills can be identified with particularly from their image. Accordingly, if the image of the remaining pills matches the image of the prescription pills, the chances are good that the remaining pills are the prescription pills. (It is possible that a patient may take the trouble to essentially manufacture pills having the same appearance as the prescription pills, but such an undertaking is difficult at best, if not essentially impossible for an individual patient lacking access to pharmaceutical packing materials and supplies.)

The system may attempt to confirm the identification of the imaged pills in different ways. In one embodiment, the system compares the image of the remaining pills to prescription pill image data to determine any matches—i.e., which prescription pill corresponds to the remaining pills. This approach, however, is computationally intensive as it requires starting with the image of the remaining pills and finding a match. Alternatively, it may be preferable, in certain embodiments, to compare the image of the remaining pills to a relative small amount of image data corresponding to the prescribed pills to determine simply if they match. By comparing the image of the pills to known image data (i.e., the image data of the prescribed pills) the determination in step 102 is reduced to whether the image matches the image data, involving a much simpler determination of yes/no or may be. In other words, in this embodiment, the system does not try and find a match of the image of the remaining pills, but determines merely if it matches the image data. For example, if the prescription is for OxyContin, then the system can obtain imaged data on OxyContin pills and compare it to the image of the remaining pills and determine whether or not the two match.

Matching the image of the remaining pills to the prescription pill image data uses known image recognition techniques, the details of which are not descried herein because they are known or obvious to one of skill in the art in light of this disclosure. Suffice to say that, in one embodiment, the image may be transformed to a numerical representation using a known transform function. This numerical representation than is compared to a numerical representation of the prescription pills to determine a match. In one embodiment, the prescription pills image data is a library of mathematical representations of prescription pills. In this respect, it may be preferable to have prescription pills image data based on different perspectives such that the onus is not on the patient to image the pill in a certain way to facilitate its identification. In another embodiment, the image of the remaining pills is not transformed but is compared to similar image data of the prescription pills. In yet another embodiment, the comparison is not made using computer recognition techniques, but rather is made manually, by a human viewing the remaining pill image data to the prescription pill image data. In such an embodiment, the human review can be performed in locations where labor costs are relatively low. In still another embodiment, a combination of computer and human recognition techniques are used (discussed below). Still other approaches for comparing images will be known to those of skill in the art.

In one embodiment, the identification of the imaged remaining pills as being the prescription pills is associated with a certainty or an estimation of accuracy. In other words, it is anticipated that in some cases the image quality or the perspective of the image may prevent an absolute confirmation that the imaged remaining pills are indeed the prescribed pills. In such a case, in one embodiment, the system provides an indication of the accuracy of the confirmation—e.g., confirmed, probably, probably not, or no, or 100% match, at least 75% match, at least 50% match, less than 50% match. As described below, the reporting of the compliance results may be a function of accuracy such that, if the accuracy drops below a certain threshold limit, an image may be flagged as requiring a follow-up. For example, in one embodiment, if the transmitted image cannot be confirmed by the system using the imaged data, then it may be forwarded to a human for human interaction/identification. Such an embodiment may be preferred when using computer recognition systems. For example, in a computer recognition system, various thresholds can be established for requiring a follow-up, manual review of the photograph—e.g., anytime the system determines a shortage of pills, a manual review can take place as a confirmation step to improve reliability. If the image is confirmed not to be the prescribed pills, then the system may proceed directly to a reporting step in which a compliance issue is raised, or, the system forwards the information to an administrator who can then contact the patient to determine whether or not a compliance issue exists requiring intervention. Still other embodiments will be obvious to those of skill in the art in light of this disclosure.

In Step 103, the number of expected pills is determined and compared with the number of remaining pills imaged. It should be understood that Step 102 and 103 can be performed in any order, or essentially simultaneously. As mentioned above, the prescription specifies the dosage of a certain type of drug to be taken in a certain period. When the image of the remaining pills is received at a given time, the system can determine the expected number of remaining pills by subtracting the number of pills that should have been consumed from the initial time the prescription was filled until the image was received. For example, if a prescription is to take one (1) pill once every eight (8) hours, and 100 pills are given on the initial date on Day 1, by Day 10, thirty (30) pills should have been consumed, and thus, seventy (70) pills should be remaining. In one embodiment, the system determines simply whether or not the correct number of pills exists. In another embodiment, the system goes further and quantifies the deficiency or excess of remaining pills.

In step 104, the system memorializes the patient's compliance and transmits this information to interested parties (e.g., the patient or the prescriber). It should be understood that, in one embodiment, the system is independent of the prescriber. Such an embodiment may be preferable for the prescriber as it relieves them of the administrative burden of monitoring compliance. Additionally, by putting a third party in charge of monitoring compliance, the third party can establish an infrastructure to efficiently monitor numerous patients and thus enjoy economies of scale. Of course, if the prescriber wants to monitor the patient directly, they are able to do so, or they can receive periodic reports or alerts as requested.

As suggested above, the reporting can be periodic, on demand, or event driven. For example, the reporting can be a periodic reporting in which the patient's compliance is transmitted to the prescriber on a periodic basis such that the physician can communicate that compliance information with the patient. Alternatively, certain triggers or alarms could be established such that the prescriber is notified of the patient's compliance, or lack thereof, when certain thresholds are met. For example, if compliance drops to a certain degree the prescriber may be notified at once. The compliance reporting can also be provided to the patient depending upon the configuration.

Referring back to FIG. 2, specific embodiments of the data stores will be considered in detail. It should be understood that although the system was depicted as being a stand-alone system, it should be understood that the system can be operated in a server-client network, peer-to-peer network, or cloud network, just to name a few. Indeed, the data stores described below can be one physical database or distributed over many databases in the cloud or other network. Additionally, the various physical data stores for storing the various data may be mixed and matched in different ways. For example, in one embodiment, the data store for pill images is one location (e.g. compliance system) and the prescription data (including initial date data) is stored in another location (e.g., the PDMP database described above). In another example, the patient and prescriber data store may be located or associated with a physician's information system, while the drug data may be associated with a pharmaceutical or national health care information system, and the compliance information may be associated with the compliance monitoring system. Still other architectures will be obvious to those of skill in light of this disclosure.

In one embodiment, the data stores comprise the following tables: patient information, patient medication, physician table, image database (resolves key to a medication), and results database. In one embodiment, the information from patient demographics, patient medication, physician table is transmitted from the doctor's office to the server from the physicians own patient management systems. This means very little data entry is necessary—the entire system is nearly automatic. However, for those doctors with older systems, a web portal can be used to enter this information into the MEDCHECK™ servers.

Patient demographics may include the following: patient id, patient name, patient address, patient email address, abuse history, plus other pertinent demographic details.

Patient medication may include, for example, medication name (i.e., OxyContin), medication id (standard usp id or other id), ailment (reason for medication), strength (i.e., 300), units of strength (milligrams), dosage (e.g. 2 pills every 8 hours), plus other pertinent details related to the patient's medication, date prescribed, date filled (i.e., initial date), date last MedCheck™ test, MedCheck™ review interval (e.g., random 7, random 15, random 30, random 90, random 180, and random 365 depending on the patient history and potency of the medication).

Physician table may include, for example, physician name, physician id, physician address, physician default MedCheck™ interval (e.g., random 7, random 15, random 30, random 90, random 180, and random 3650).

The medication image database may include, for example, medication name (i.e., OxyContin), medication id (standard usp id or other id), various perspectives of the medication image keys (e.g., a unique id created by the pattern scanning/analysis algorithm for a single tablet front, back, side), plus any other image keys required from various viewing angles of the tablet.

MedCheck™ results table may include, for example, transaction id, patient id, medication id, image key [1 . . . x], quantity pills determined, raw image Qpeg) pathname/filename, date of scan, expected number: (based upon last prescription (always known) or refill (if known)), imaged number, results (e.g., quantity found=quantity expected [normal], quantity found>quantity expected [abnormal], quantity found<quantity expected [abnormal], quantity found<<quantity expected [critical] quantity found<<quantity expected [critical], indeterminate [abnormal], refused/nonresponsive/uncooperative [abnormal]), date physician notified (e.g., critical (when marked, notify physician immediately.)

### Example

First, the system interrogates the patient medication table and extracts all records that have not been tested within the timeframe of DATE-LAST-MEDCHECK-TEST and MEDCHECK INTERVAL. In this example, the MEDCHECK INTERVAL is random 7, and the DATE-LAST-MEDCHECK-TEST indicates the last test was 8 days ago, accordingly, the system initiates a test.

A link is made to the patient demographic table to obtain the email address of the patient. The patient is sent an email explaining they must take a picture of their medication, possibly arranged in a particular fashion (square, diamond, circle, rows, columns, etc.) The patient emails his image to the system. The subject line conveys the transaction ID and this is used to identify the patient's image of remaining pills when received.

A server opens the email, collects the transaction ID from the subject line, and then an image recognition algorithm SCANS the picture. Each tablet (pill) can be identified by a pattern scanning and image analysis algorithm to produce a “KEY.” Since we know what the patient should be taken, it is far easier to determine the medication based on KEY. If the probably of identification drops below a certain level, the system may send for manual review. This results in creation of the MEDCHECK™ results record. The medication KEY is entered and identified to a MEDICATION ID. Next, the number of pills discerned is updated in this same record.

Next, a determination is made, based on the medication table to see how many pills are expected and this is entered into the results record as well. If the critical flag is checked, and the number of found pills is out of spec with the expected number of pills, the physician is notified immediately. If the critical flag is not checked, the current process or a future process can scan all of the results records and send a response to the physician's patient management system. In turn, the final results are placed electronically into the patient's chart (by the doctor's patient management system). The next time the patient sees his or her doctor, the physician can use this information (historical information is valuable too) to make sure the patient is using his medication properly. In cases where the medication cannot be determined (e.g., poor lighting in photo), but the number of pills can be ascertained, a manual entry in the results file is made. Plus, a human can confirm the medication type and set that information in the results record as well.

The entire process can work silently without the involvement of the physician. The physician only becomes involved the next time the doctor sees the patient.

It should be understood that the foregoing is illustrative and not limiting and that obvious modifications may be made by those skilled in the art without departing from the spirit of the invention. Accordingly, the specification is intended to cover such alternatives, modifications, and equivalence as may be included within the spirit and scope of the invention as defined in the following claims.

What is claimed is:

1. A method of determining a patient's compliance with a prescription, said prescription indicating the dosage of prescription pills to be consumed in a certain period, said method comprising:
(a) receiving an image of one or more remaining pills from a patient on a given date, said given date being after an initial date when said patient received an initial number of said prescription pills;
(b) confirming that said remaining pills are the same type as said prescription pills;
(c) determining whether the number of said remaining pills corresponds with an expected number of remaining pills, said expected number of remaining pills being said initial number minus a calculated number of pills taken between said initial date and said given date based on said dosage; and
(d) generating a report of said patient's compliance based on whether said number of said remaining pills corresponds to said expected number of remaining pills as determined in step (c).

2. The method of claim 1, wherein said report is provided to the prescriber of said prescription.

3. The method of claim 1, wherein said report comprises a degree of compliance.

4. The method of claim 3, wherein said report comprises an estimation of accuracy of results.

5. The method of claim 4, wherein said report is provided to the prescriber of said prescription if said degree of compliance reaches a predetermined point.

6. The method of claim 1, wherein said report is provided to said patient.

7. The method of claim 1, wherein step (a) is scheduled to occur periodically.

8. The method of claim 1, wherein step (a) is initiated randomly by said prescriber.

9. The method of claim 1, wherein step (a) is initiated by said patient to determine if said patient should consume one or more pills on said given date to be compliant with prescription.

10. The method of claim 1, wherein steps (b) and/or (c) are performed using a computer.

11. The method of claim 10, wherein step (b) is performed using an image recognition system.

12. The method of claim 11, wherein steps (b) and (c) are performed using said image recognition system.

13. The method of claim 1, wherein step (a) comprises receiving an image from a patient's wireless device.

14. The method of claim 13 wherein said wireless device is a smartphone or a tablet.

15. The method of claim 1, wherein said (a) comprises receiving an image from a kiosk in a public area.

16. The method of claim 1, further comprising: transmitting to said patient a request for a shape on said given date, and wherein step (a) comprises said patient arranging said pills in said shape prior to taking said image of said pills.

17. The method of claim 1, wherein said image comprises a date stamp.

18. A system for monitoring compliance comprising:
at least one processor;
one or more data stores operatively connected to the processor;
a network interface to transmitting requests and reports; and;
memory operatively connected to the process and configured with instructions for causing the processor to execute the following steps:
(a) receiving an image of one or more remaining pills from a patient on a given date, the given date being after an initial date when the patient received an initial number of the prescription pills;
(b) confirming that the remaining pills are the same type as the prescription pills;
(c) determining whether the number of the remaining pills corresponds with an expected number of remaining pills, the expected number of remaining pills being the initial number minus a calculated number of consumed pills based on the dosage taken between the initial date and the given date; and
(d) generating a report of the patient's compliance based on whether the number of the remaining pills corresponds to the expected number of remaining pills as determined in step (c).

19. A device comprising:
a camera for taking an image of pills;
an network interface for transmitting said image over a network to a compliance system; and
a user interface configured to instruct the user when to take an image of said pills.

20. The device of claim 18 wherein said user interface is configured to communicate with said compliance system through said network interface to determine when said user should take said image.

21. The device of claim 19, wherein said user interface also instructs the user how to arrange the pills in the image.
